(19) United States
(12) Patent Application Publication
(10) Pub. No.: US 2024/0108492 A1
(43) Pub. Date: Apr. 4, 2024

(54) An Ostomy Collection Device

(71) Applicant: Salts Healthcare Limited, Birmingham, West Midlands (GB)

(72) Inventors: Marcus Allen, Birmingham, West Midlands (GB); Mark Heynes, Stafford (GB); John Harveson, Stafford (GB); William Morris, Stafford (GB)

(21) Appl. No.: 18/274,522

(22) PCT Filed: Feb. 3, 2022
(86) PCT No.: PCT/GB2022/050284

§ 371 Date: Jul. 27, 2023

(30) Foreign Application Priority Data
Feb. 5, 2021 (GB) 2101649.8

(51) Int. Cl. A61F 5/445; A61F 5/44

(52) U.S. Cl. CPC A61F 5/445; A61F 5/4407

(57) ABSTRACT

We provide an ostomy collection device including: a housing defining a collecting volume for receiving and storing waste from an ostomy appliance, the housing including an inlet for fluidly connecting to an outlet of an ostomy appliance so that waste from the ostomy appliance flows into the collecting volume; and a cover layer positioned over an external surface of the housing.

### DESCRIPTION OF INVENTION

This invention relates to an ostomy collection device, and in particular but not exclusively to an ostomy collection device for use with a urostomy appliance.

Ostomy appliances are well known. Typically, a person having a stoma uses an ostomy appliance to collect waste from their stoma. The ostomy appliances collects a volume of waste and, once full or nearing full, the user either changes the appliance for a fresh one or empties the waste from the existing appliance.

This process works well while the user is able to visit a suitable space for changing/emptying their ostomy appliance—e.g. a bathroom or toilet. However, there may be times when the user does not wish to empty or change their appliance as often, for example, when travelling or while asleep.

The current solution is to provide the user with a flexible bag which may be supported from a structure/frame. The bag is connected to the user's ostomy appliance to increase the volume of waste that can be collected. However, the frames to which the bag is attached can be unstable and are easily knocked over.

Embodiments of the present invention seek to address one or more problems associated with the prior art.

According to a first embodiment we provide an ostomy collection device including:
a housing defining a collecting volume for receiving and storing waste from an ostomy appliance,
the housing including an inlet for fluidly connecting to an outlet of an ostomy appliance so that waste from the ostomy appliance flows into the collecting volume; and
a cover layer positioned over an external surface of the housing.

Further aspects of the invention are set out in the appended set of numbered claims.

Embodiments of the present invention are described, by way of example only, with reference to the accompanying drawings, in which:

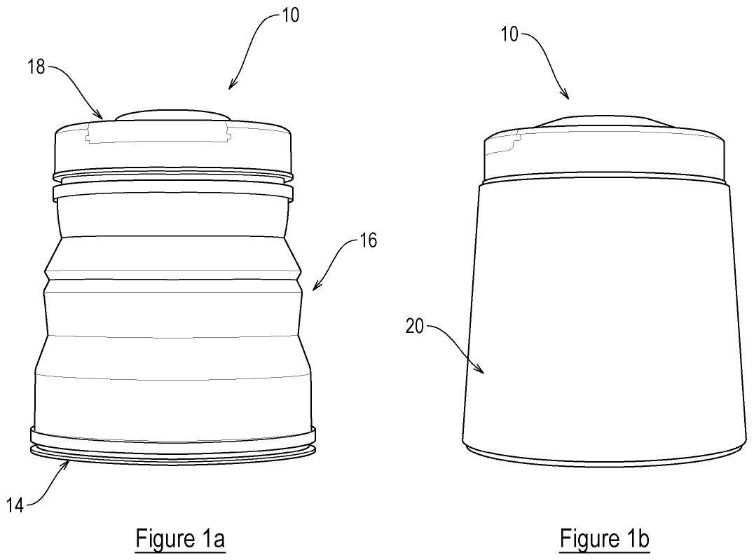

FIGS. 1a and 1 b illustrate an ostomy collection device in accordance with an embodiment of the present invention,

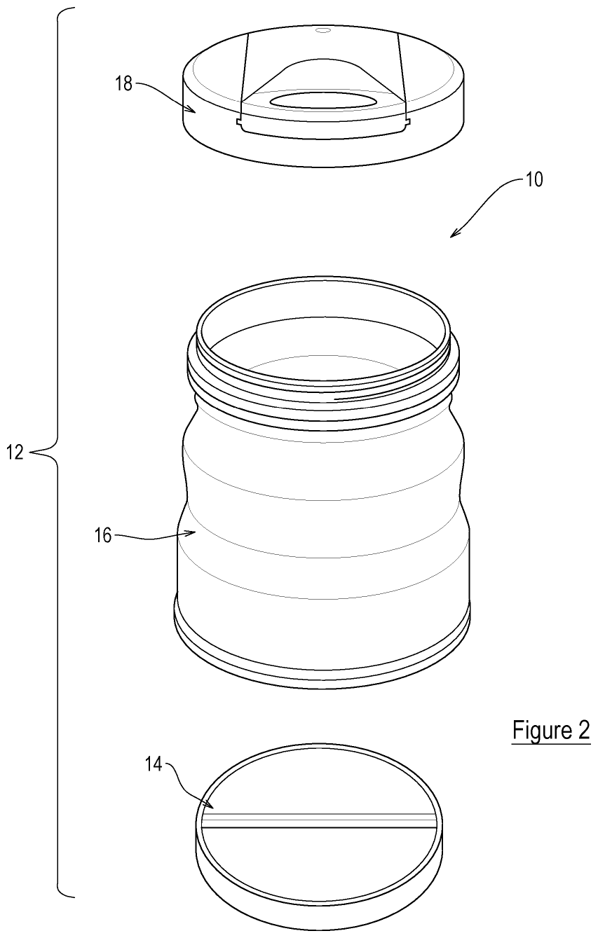

FIG. 2 is an exploded view of the ostomy collection device of FIG. 1a,

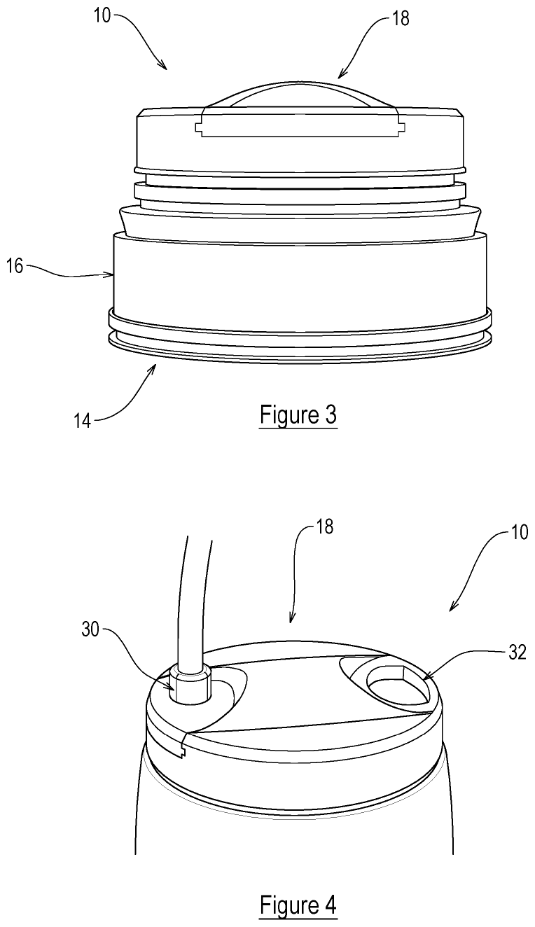

FIG. 3 illustrates an ostomy collection device in a retracted state,

FIG. 4 is a perspective view of a part of the ostomy collection device,

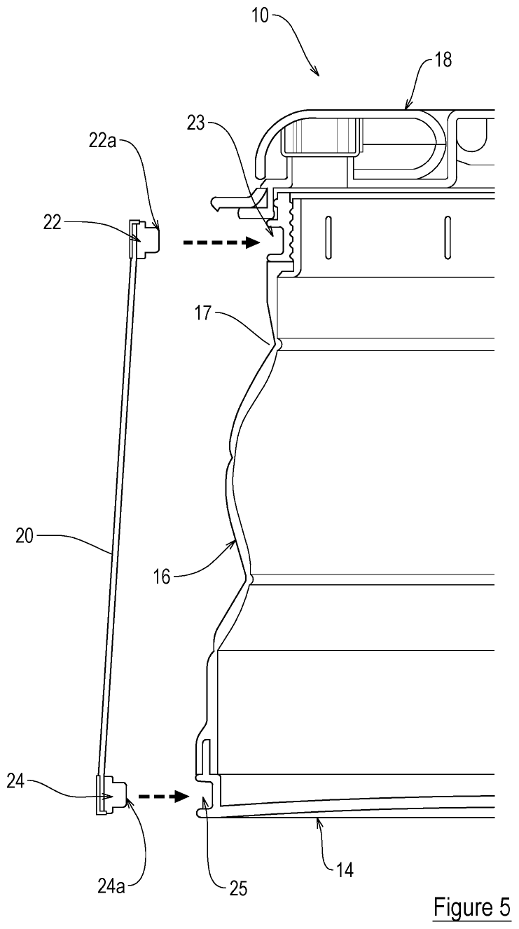

FIG. 5 is a cross-sectional view of a part an ostomy collection device,

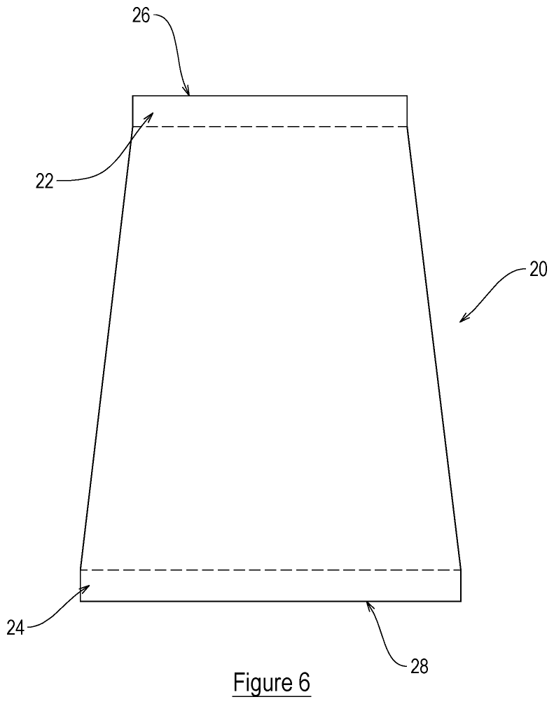

FIG. 6 is a side view of a part of the ostomy collection device.

An ostomy collection device 10 is shown in FIGS. 1a and 1b. The ostomy collection device 10 is configured to connect to an outlet of an ostomy appliance. In use, the ostomy appliance connects about a user's stoma (and collects waste exiting the stoma).

The ostomy collection device 10 includes a housing 12, which defines a collecting volume for receiving and storing waste from an ostomy appliance. The housing includes an inlet 30 and an outlet 32. The inlet 30 is configured to connect to an outlet of an ostomy appliance (not shown), so that waste from the ostomy appliance flows into the collecting volume. The outlet 32 allows the contents of the collecting volume to be disposed of (e.g. into a toilet, etc.).

In some embodiments, the inlet 30 and the outlet 32 are both positioned on a top 18 of the housing 12.

In some embodiments (for example, as shown in FIGS. 1a and 1b), the housing 12 has a base 14, a wall 16 and a top 18. In the present example, the wall 16 extends between the base 14 and the top 18 to enclose the entire collecting volume.

The base 14 supports the ostomy collection device 10 when positioned on a surface, such as a floor or table, etc. The base 14 provides a substantially flat portion to the ostomy collection device 10 (to provide stability and reduce the risk of the device 10 being knocked over). The base 14 and top 18 are (when viewed from directly above the housing 12) substantially circular (although it should be appreciated that other shapes are also possible and the shapes of the base 14 and the top 18 do not have to match necessarily).

In embodiments, the wall 16 is connected to the base 14 (for example, the wall 16 is connected around a peripheral edge of the base 14). In some embodiments, the wall 16 and the base 14 are permanently connected. In some embodiments, the base 14 is disconnectable/disengageable from the wall 16 (in such an example, the base 14 and the wall 16 include respective engagement formations, which engage with each other so that the base 14 connects to the wall 16). In some embodiments, the engagement formations are corresponding threaded portions. In other words, the base 14 has an engagement formation which incorporates a helical thread and the wall 16 also has an engagement formation which incorporates a helical thread. On relative rotation of the two helical threads together, the base 14 and the wall 16 are connected together.

In embodiments, the wall 16 extends substantially upwards from the base 14 to the top 18 (and is connected to the top 18). In some embodiments, the wall 16 and top 18 are permanently connected. In some embodiments, the top 18 is disconnectable/disengageable from the wall 16 (as above, in such an example, the base 18 and the wall 16 include respective engagement formations, which engage with each other so that the base 14 connects to the wall 16). In some embodiments, the engagement formations are corresponding threaded portions. In other words, the top 18 has an engagement formation which incorporates a helical thread and the wall 16 also has an engagement formation which incorporates a helical thread. On relative rotation of the two helical threads together, the top 18 and the wall 16 are connected together.

In embodiments, there is a single continuous wall 16, which extends around the entire periphery of the base 14 and the top 18 to create an internal volume defined by the base 14, the wall 16 and the top 18. In the illustrated examples, the wall 16 is substantially cylindrical in shape, e.g. it is ring-shaped or annular. The wall 16 may be constructed from multiple portions connected together, if desired, but still provide generally cylindrical enclosure.

In embodiments, the wall 16 is constructed from a deformable material, which allows the wall 16 to flex and change shape. In some embodiments, this material is an elastomer, such as silicone. In some embodiments, the wall 16 is constructed from a single material (but it may be made from a plurality of different materials).

In the present example, the width of the base 14 is larger than the width of the top 18, which allows the ostomy collection device 10 to be more stable when positioned on a surface. It should be appreciated that the top 18 may be wider than the base or the top 18 and the base 14 may be substantially the same width.

In some embodiments, the volume of the housing 12 is changeable depending on the state of the ostomy collection device 10. The ostomy collection device has at least two different states, each having a different internal volume. In the first state, the housing 12 of the ostomy collection device 10 is compressed or retracted and the internal volume of the housing 12 has a first value. In the second state, the housing 12 is extended or uncompressed and the internal volume of the housing 12 has a second value. In some embodiments, the ostomy collection device 10 has additional states, which relate to partially-extended or partially-compressed positions, in which states the internal volume lies between the first and second values.

In some embodiments, the wall 16 is ridged or concertina-shaped (this allows the wall 16 to fold upon itself). As shown in FIG. 3, the ridged or concertinaed wall 16 of the ostomy collection device 10 may be manipulated so that at least a portion of the wall 16 is folded upon itself. In this configuration, the housing 12 is in a compressed or retracted state (e.g. the first state). It should be appreciated that when the wall 16 is wholly/completely collapsed, the internal volume of the housing 12 and the height of the housing 12 is minimised. As shown in FIG. 1a, the ridged or concertinaed wall 16 may be substantially straightened to provide the extended or expanded state.

When the wall 16 is completely expanded, the housing 12 has its largest volume and tallest height. In the described example, the first value (i.e. the first state housing 12 volume) is smaller than the second value (i.e the second state housing 12 volume). The move to the expanded state increases the internal volume of the housing 12 of the ostomy collection device 10 relative to its compressed or retracted state.

The wall 16 in the illustrated embodiments has multiple folding positions provided and as such intermediate states are provided because the wall 16 may not be completely compressed or completely expanded—providing an intermediate housing 12 volume and height (a user may choose to use these intermediate stable positions of the walls 16 if desired).

The possible configurations, and thus possible internal volumes, of the housing 12 are dependent on the construction of the wall 16. With reference to the figures, the concertina-shaped wall 16 includes a number of faces or surfaces. In other words, the wall 16 includes a number of folding positions 17. The folding positions 17 are provided by parts of the wall 16 having different radiuses to other parts. These different parts provide natural folding positions along the wall 16.

The folding positions 17 could also be provided by material of a different thickness (for example, it may be thinner than other parts of the wall 16) or a combination of the two mechanisms. This allows the wall 16 to be manipulated reliably and/or repeatedly into a number of predetermined configurations/internal volumes.

In some embodiments, the wall 16 may be made of a combination of materials. The folding positions 17 may be constructed from a material which is more flexible than the faces or surfaces of the wall 16. It should be appreciated that the folding positions may be provided by an intersection or the like between two materials.

In some embodiments, the number of possible configurations, and thus possible internal volumes of the housing 12 of the ostomy collection device 10, depends on the number of joints 17 present on the wall 16.

It should be appreciated that the wall 16 may be shaped or constructed in such a way that allows the internal volume to be altered by twisting the housing 12 of the ostomy collection device 10. In some embodiments, the ostomy collection device 10 includes a securing mechanism (not shown) which aids in maintaining the housing 12 in a configuration (e.g. a toggle and loop fastener mechanism, straps, hook and loop fasteners, magnets or any other appropriate mechanisms).

In some embodiments, the ostomy collection device 10 includes a biasing mechanism (not shown) for biasing the housing 12 towards a specific configuration or configurations, for instance by the use of a resilient element (such as a spring). In some embodiments, the ostomy collection device 10 includes a securing mechanism and a biasing mechanism, or only one of a securing mechanism and a biasing mechanism.

The ostomy collection device 10 includes a cover layer 20, which is positioned over an external surface of at least a part of the housing 12. The cover layer covers an external surface of at least a portion of the housing 12. In the example shown in FIG. 1b, the cover layer 20 extends over the entirety of the wall 16 (while the top 18 and base 14 are exposed). It should be appreciated that the cover layer 20 may partially or wholly cover other parts of the housing 12 (e.g. such as the base 14 or the top 18).

In some embodiments, the cover layer 20 is shaped to correspond with the shape/profile of a part of the housing 12. For example (shown FIG. 1b), the cover layer 20 is shaped similarly to the housing 12.

In some embodiments (see for example the illustration in FIG. 6), the cover layer 20 has a top edge 26 and a bottom edge 28. In embodiments, the top edge 26 of the cover layer 20 is positioned at or near the top 18 of the housing 12 and the bottom edge 28 of the cover 20 is positioned at or near the base 14 of the housing 12 (shown in FIG. 5).

In some embodiments, the cover layer 20 is formed of a fabric material, which may be a woven or non-woven fabric material. In some examples, the cover layer 20 is elasticated, which allows the cover 20 to conform to at least a portion of the housing 12. In other words, the cover layer 20 is stretched to adopt the form and/or dimensions of the housing 12.

In embodiments, as the cover layer 20 is made from a fabric material, or otherwise flexible material, the shape of the cover layer 20 may change or be altered when the housing 12 is configured between states defining different internal volumes. For instance, as shown in FIG. 1b, when the housing 12 is in an extended state, the cover layer 20 is extended so as to cover the wall 16. In this instance, the cover layer 20 is drawn out over the wall 16 with little or no folding (e.g. the cover layer 20 is taut over the housing 12).

Conversely, when the housing 12 is in the first (compressed) state, the cover layer 20 folds or otherwise changes shape or form, so as to conform in shape to the housing 12. In this instance, the cover layer 20 may fold, or form a series of ripples, or may take any other appropriate form.

In embodiments, the cover layer 20 is removably connected to the housing 12. To facilitate attachment and removal from the housing 12, the cover layer 20 includes a first engaging formation 22 and the housing 12 includes a corresponding first engaging formation 23. The first engaging formations 22, 23 interact/cooperate to attach the cover layer 20 to the housing 12.

In some embodiments, the first engaging formation 22 is located adjacent or near the top edge 26 of the cover layer 20 and located on a surface of the cover layer 20 which faces the external surface of the wall 16 (shown in FIGS. and 6). The corresponding first engaging formation 23 is located on the top 18 of the housing 12 (or adjacent/near the top 18).

In embodiments, the first engaging formation 22 includes a protrusion 22a. In embodiments, the first engaging formation 22 is a continuous ridge/flange/rim, which extends around the entire top edge 26 of the cover 20, or it may be of a plurality of pieces which each extend around a portion of the top edge 26 of the cover 20. In embodiments, the corresponding first engaging formation 23 is a continuous recess that extends around the entire periphery of the housing 12, or it may be of a plurality of recesses which extend around a portion of the periphery of the housing 12.

In some embodiments, the first engaging formation 22 and corresponding engaging formation 23 includes other attachment mechanisms such as hook and loop fasteners, magnets, adhesives or any other mechanisms which allow the cover layer 20 to be attached to the housing 12.

In some embodiments (see FIG. 5), the cover layer 20 includes a second engaging formation 24, which may or may not be similar in construction to the first engaging formation 22. The second engaging formation 24 is located adjacent or near the bottom edge 28 of the cover layer 20 and located on a surface of the cover layer 20 which faces the external surface of the wall 16. The housing 12 may include a corresponding engaging formation 25 which is adapted to engage or otherwise receive or attach to the second engaging formation 24. The corresponding engaging formation 25 may be located on the base of the housing 12, or a part of the housing 12 which is adjacent or near the base 14.

An ostomy collection device 10 that includes a cover layer 20 is advantageous in that it serves to conceal the contents of the housing 12 when in use, or that it may disguise the housing 12 (e.g. the cover layer 20 disguises the appearance of the housing 12, and therefore the ostomy collection device 10, so that people other than the user are not able to distinguish it as an ostomy collection device 10). This enhances the user's sense of discretion and/or privacy when using the ostomy collection device 10, or when it is otherwise visible.

The cover layer 20 may protect the housing 12 of the ostomy collection device (e.g. the cover layer 20 may protect the housing 12 from impact, sharp objects and undesirable substances). Furthermore, a removable cover layer 20 may be replaced in the event that it becomes damaged, or if the user wishes to change the outward appearance of the housing 12 of the ostomy collection device 10.

Representative features are set out in the following clauses, which stand alone or may be combined, in any combination, with one or more features disclosed in the text and/or drawings of the specification.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

Although certain example embodiments of the invention have been described, the scope of the appended claims is not intended to be limited solely to these embodiments. The claims are to be construed literally, purposively, and/or to encompass equivalents.

1. An ostomy collection device including:
a housing defining a collecting volume for receiving and storing waste from an ostomy appliance,
the housing including an inlet for fluidly connecting to an outlet of an ostomy appliance so that waste from the ostomy appliance flows into the collecting volume; and
a cover layer positioned over an external surface of the housing.

2. An ostomy collection device according to claim 1 wherein the cover layer is removable.

3. An ostomy collection device according to claim 1 wherein the cover layer is formed from a fabric material.

4. An ostomy collection device according to claim 3 wherein the cover layer is formed from a woven or non-woven fabric material.

5. An ostomy collection device according to claim 3 wherein the cover layer material is elasticated.

6. An ostomy collection device according to claim 1 wherein the cover layer includes a first engaging formation and the housing includes a corresponding first formation for attaching the cover layer to the housing.

7. An ostomy collection device according to claim 6 wherein the cover layer includes a second engaging formation and the housing includes a corresponding second formation such that the cover layer can be attached to the housing.

8. An ostomy collection device according to claim 1 wherein, in use, the housing is configurable between a retracted state wherein the collection volume is at a first value, and an expanded state wherein the collection volume is at a second value.

9. An ostomy collection device according to claim 8 wherein the first value is smaller than the second value.

10. An ostomy collection device according to claim 8 wherein, in use, configuring the housing between states causes the at least a portion of the cover layer to substantially adapt to the shape of the housing.

11. An ostomy collection device according to claim 1 wherein the housing includes:
a base configured to support the device when positioned on a surface,
a top, and
a wall extending between the base and the top.

12. An ostomy collection device according to claim 11 wherein the cover layer covers the entirety of the wall.

13. An ostomy collection device according to claim 11 wherein the wall is substantially cylindrical.

14. An ostomy collection device according to claim 11 wherein the width of the housing at or near the top is less than the width of the housing at or near the base.

15. An ostomy collection device according to claim 11 wherein the wall is formed from a deformable material, and optionally the material is an elastomer material, and preferably silicone.

16. An ostomy collection device according to claim 11 wherein at least a portion of the wall is substantially concertina shaped.

17. An ostomy collection device according to claim 11 wherein the top is disconnectable from the wall.

18. An ostomy collection device according to claim 1 further including an outlet which is separate from the inlet for waste to be emptied from the collection volume.
